(19) United States
(12) Patent Application Publication
(10) Pub. No.: US 2015/0272919 A1
(43) Pub. Date: Oct. 1, 2015

(54) PHARMACEUTICAL COMPOSITION

(71) Applicant: DIPHARMA FRANCIS S.R.L., Baranzate (IT)

(72) Inventors: Franca Ferrari, Pavia (IT); Silvia Rossi, Pavia (IT); Maria Cristina Bonferoni, Pavia (IT); Giuseppina Sandri, Pavia (IT); Carla Caramella, Pavia (IT); Pietro Allegrini, Baranzate (IT)

(73) Assignee: Dipharma Francis S.r.l., Baranzate (IT)

(21) Appl. No.: 14/432,773

(22) PCT Filed: Sep. 19, 2013
(86) PCT No.: PCT/EP2013/069461

§ 371 Date: Apr. 1, 2015

(30) Foreign Application Priority Data
Oct. 3, 2012 (IT) MI2012A001654

(51) Int. Cl. A61K 31/21; A61K 47/26; A61K 47/32

(52) U.S. Cl. CPC A61K 31/21; A61K 47/32; A61K 47/26

(57) ABSTRACT

Pharmaceutical compositions useful in the treatment of sexual and urogenital dysfunctions such as dyspareunia, vulvodynia, vaginismus, vestibulitis and anal fistulas.

### FIELD OF INVENTION

The present invention relates to pharmaceutical compositions containing nitroglycerin which are useful in the treatment of sexual and urogenital dysfunctions such as dyspareunia, vulvodynia, vaginismus and vestibulitis, and anal fistulas. The invention also relates to the process for preparing said compositions.

### PRIOR ART

Disorders of the urogenital apparatus, such as dyspareunia, vulvodynia, vaginismus and vestibulitis, are unfortunately quite common. For example, about 15-19% of women of fertile age and up to 33-39% of post-menopausal women are known to suffer from these disorders.

The etiology of said disorders is not yet fully understood, but it is believed to be multifactorial and multisystemic, and that biological (infectious, inflammatory, microtraumatic, myalgic, hormonal and hyperalgesic), psychosexual and/or relational causes contribute to their onset.

Other disorders, such as anal fistulas, are also very common and unpleasant, and affect both sexes at all ages.

As regards pharmacological treatment, a number of pharmaceutical formulations for topical use have been studied.

Among the various compositions for topical use, formulations in cream or ointment form containing N-oxide donors such as nitroglycerin have proved very promising.

Nitroglycerin-based ointments such as RECTOGESIC®, containing 0.4% w/w nitroglycerin, are present on the market, which are used to treat anal fistulas and rhagades, but can also be used to treat sexual dysfunctions such as dyspareunia.

Unfortunately, in view of the extensive vascularisation of the treatment site, said compositions present the drawback of causing systemic effects such as headaches. Moreover, the ointment formulation causes low residence in situ of the product, is inconvenient to use, and the treatment is not standardisable.

For this reason, the inventors of the present invention have studied in depth a method of preparing a pharmaceutical formulation that is suitable for the treatment of said urogenital disorders and eliminates said drawbacks.

It has surprisingly been found that particular sponge-like compositions, in the form of a lyophilisate, containing specific quantities of nitroglycerin, enable an accurate dose of the medicament to be obtained, with easy administration and immediate release thereof, and at the same time eliminate the adverse systemic effects reported above.

In their study, the inventors of the present invention also had to deal with formulation problems, mainly associated with the short residence time of the active ingredient nitroglycerin in the pharmaceutical form and its migration towards the packaging material of the formulation, especially if said material is made of plastic (elastomer).

These formulation problems are mainly due to the volatility of the active ingredient; see, for example: Richman M. D., Fox C D., Shangraw R. F. “Preparation and stability of glyceryl trinitrate sublingual tablets prepared by direct compression”. J. Pharm. Sci. 54, 447 (1965); and Pikal M J, Lukes A L, Conine J W., “Effect of nitroglycerin-soluble additives on the stability of molded nitroglycerin tablets”. J Pharm Sci. 73, 1608 (1984).

In view of the major problems involved in preparing the formulation, mainly due to the high volatility of nitroglycerin, sponge-like formulations containing nitroglycerin have never been studied for the disorders described above.

In particular, the preparation of pharmaceutical formulations in lyophilisate form containing nitroglycerin is difficult due to the technical problems involved in the lyophilisate preparation process. Freeze-drying involves cryodrying of liquid formulations under high vacuum which, as well as removing the solvent, causes high levels of loss of the active ingredient during the sublimation (primary drying) and desorption (secondary drying) stages.

There is consequently a need for a pharmaceutical composition for clinical use in the form of a lyophilisate containing nitroglycerin, which does not cause systemic effects, or at least minimises them, is easily applicable, has a good residence time in situ, allows dose standardisation and is easy to prepare industrially.

It has surprisingly been found that the compositions according to the present invention in lyophilisate form eliminate the drawbacks illustrated above. In particular, the compositions according to the invention maintain the nitroglycerin content of the formulation during the various preparation stages, and possess particular mechanical strength and hydration properties.

### SUMMARY OF THE INVENTION

The invention relates to a sponge-like composition in the form of a lyophilisate, comprising a therapeutically effective quantity of nitroglycerin, a mixture containing crosslinked polyvinylpyrrolidone and non-crosslinked polyvinylpyrrolidone, a cryoprotectant and, if appropriate, an excipient and/or carrier, its use in the treatment of sexual and/or urogenital dysfunctions, and a process for its preparation.

### DETAILED DESCRIPTION OF THE INVENTION

The subject of the present invention is a pharmaceutical composition, preferably in the form of a lyophilisate, comprising:

nitroglycerin ranging from about 0.04 to about 0.3% w/w;

a mixture of crosslinked and non-crosslinked polyvinylpyrrolidone, ranging from about 30 to about 70% w/w; and

a cryoprotectant or stabilising agent ranging from about 20 to about 60%.

According to the present invention, a crosslinked polyvinylpyrrolidone is, for example, crospovidone, preferably a PVP XL 10, while a non-crosslinked polyvinylpyrrolidone is a high-molecular-weight polyvinylpyrrolidone, typically ranging between about 1,100,000 and about 1,500,000 daltons, preferably around 1,300,000 daltons, typically PVP 90.

In said mixture the weight ratio between crosslinked polyvinylpyrrolidone and non-crosslinked polyvinylpyrrolidone is, for example, between about 1:2 and about 1:4. According to a preferred aspect of the invention, said mixture comprises a PVP XL 10 and PVP 90, XL 10 (ISP); typically in a ratio of between about 1:2.2 and about 1:3.7. A PVP XL 10 can, for example, be the one marketed as Polyplasdone® XL 10 (ISP); a PVP 90 can, for example, be the one marketed as Kollidon® 90 (BASF).

According to the present invention, a cryoprotectant is an agent used in the freeze-drying process which aids said freeze-drying process and maintains the stability characteristics of the lyophilisate.

A cryoprotectant or stabiliser can be a polyalcohol, such as mannitol, sorbitol, xylitol, maltitol or lactitol; a monosaccharide such as glucose; a disaccharide such as sucrose, trehalose or lactose; a polysaccharide such as dextran; an aminoacid, such as histidine; or polyethylene glycol.

According to a preferred aspect of the invention a cryoprotectant or stabilising agent is mannitol or sorbitol, more preferably mannitol.

The compositions according to the invention can optionally contain a further pharmaceutically acceptable excipient and/or carrier. Said excipient and/or carrier can be one or more ingredients selected, for example, from the group comprising water, preferably bidistilled water; glycine, gelatin, fructose and inositol; a stabilising agent, such as a lyoprotectant, for example a water-soluble branched polymer of high molecular weight synthesised from sucrose and epichlorhydrin, in particular Ficoll®; a chelating agent such as EDTA; a preservative; an antioxidant; an isotonic agent such as sodium chloride; a buffer system, such as citrate, phosphate, tromethamine, succinate or lactate; an inorganic salt, such as sodium chloride; a surfactant, such as polysorbate 80; or a mixture of two or more thereof.

According to a preferred aspect of the invention the pharmaceutical composition according to the invention, preferably in the form of a lyophilisate, comprises:

nitroglycerin from about 0.05 to 0.28, preferably from about 0.24 to about 0.28% w/w;

PVP 90 from about 35.0 to about 45.0% w/w;

PVP XL 10 from about 10.0 to about 18.0% w/w; and

mannitol from about 40.0 to about 52.0% w/w.

According to a particularly preferred aspect of the invention, the freeze-dried pharmaceutical composition according to the invention is in single-dose form containing from about 50 to about 200 μg of nitroglycerin (GTN).

The pharmaceutical composition can be prepared, for example, in the form of substantially cylindrical sponges, typically having a diameter of about 1.2 cm, a height of about 0.8 cm, a weight ranging between about 54 mg and 110 mg, and a nitroglycerin concentration ranging between about 0.05 and about 0.3%.

According to a further aspect thereof, the invention provides a process for the preparation of a pharmaceutical composition in the form of a lyophilisate, as defined above, comprising:

a) adsorbing nitroglycerin on a grade of solid crosslinked polyvinylpyrrolidone;

b) adding bidistilled water to the mixture prepared in step a);

c) adding to the aqueous suspension obtained in step b) of volumes of aqueous solutions containing non-crosslinked polyvinylpyrrolidone and a cryoprotectant respectively; and

d) freeze-drying of the mixture thus obtained.

Typically, said pharmaceutical composition can be obtained using the following materials:

nitroglycerin diluted to about 5% w/w in a C₂-C₅ glycol, in particular propylene glycol;

a solid crosslinked polyvinylpyrrolidone, typically crospovidone, preferably PVP XL 10;

a non-crosslinked polyvinylpyrrolidone, preferably PVP 90, in aqueous solution from about 5 to 7% w/w, preferably about 6% w/w;

a cryoprotectant, preferably mannitol in aqueous solution from about 8% to 13% w/w, preferably about 10% w/w; and

bidistilled water.

According to a preferred method of embodiment of the invention, the process for preparation of the pharmaceutical composition can be performed as follows:

nitroglycerin (GTN), diluted to 5% w/w in propylene glycol, is adsorbed on solid crosslinked polyvinylpyrrolidone, preferably PVP XL 10, for between about 8 and 20 minutes, preferably about 10 minutes.

Solid PVP XL 10 is preferably used, already positioned in a 5 ml amber glass vial, which is then employed for the freeze-drying process, having been stoppered immediately after the addition of the medicament.

Solid PVP XL 10 is used in a quantity sufficient to obtain a suspension of about 10% (w/w) in PVP XL 10 at the end of the adsorption stage, after the addition of bidistilled water to the mixture with nitroglycerin.

Volumes of aqueous solutions of non-crosslinked polyvinylpyrrolidone, typically having high molecular weight, preferably 6% (w/w) PVP 90 and 10% (w/w) mannitol, are added to said suspension. Said volumes are added in suitable quantities, as exemplified in the experimental section, with a view to optimising the technical characteristics of the pharmaceutical formulation due to the high volatility of nitroglycerin.

The mixture thus obtained can be freeze-dried by methods known to the prior art. Typically, the suspension thus formed is frozen at −20° C. overnight and freeze-dried (Heto Drywinner, Analitica de Mori, Milan) for 24 h.

By operating as reported above and introducing a suitable quantity of suspension into each vial, a sponge-like formulation is obtained which has a diameter of about 1.2 cm, a height of about 0.8 cm, and a weight ranging between about 54 mg and 110 mg.

A further subject of the invention is a pharmaceutical composition as obtainable according to the process illustrated above.

The pharmaceutical composition according to the invention can be usefully applied in the treatment of sexual and urogenital dysfunctions such as dyspareunia, vulvodynia, vaginismus and vestibulitis, and anal fistulas in patients who need such treatment.

The term “treatment” means the use of the composition to treat said disorders and alleviate the patient's pain and discomfort.

As reported above, the compositions according to the invention maintain the nitroglycerin content in the formulation through the various stages of its preparation, and possess special mechanical strength and hydration properties. This is demonstrated by the following analysis methods.

Analysis of Nitroglycerin Content in Sponges

After freeze-drying, the nitroglycerin (GTN) content of the sponges is determined by HPLC analysis according to the specifications set out in the USP 34 monograph “Diluted nitroglycerin”.

The residual % of nitroglycerin in the samples tested exceeds 90%.

Mechanical Strength Properties

The mechanical strength measurements in the dry state are conducted with a texture analyser (TA.XTplus Texture Analyser, Stable Microsystem, UK) equipped with a 5 Kg load cell. A cylindrical probe with a diameter of 50 mm is inserted into the sponge for a length of 3 mm at the rate of 10 mm/sec.

The penetration resistance (F), namely the force required to insert the probe into the sample, and the corresponding movement values up to a maximum distance of 3 mm, are recorded on computer and subsequently processed with specific software (Exponent, Stable Microsystem, UK).

The penetration energy (L), corresponding to the area under the curve of penetration resistance vs. the distance travelled by the probe, is calculated from the said pairs of data. Three tests are performed for each sponge.

The penetration energy is greater than 15 N mm.

Hydration Properties

The hydration properties of the sponges are evaluated in terms of volume and dissolution time in a pH 4.5 acetate buffer.

Dissolution Volume

Aliquots of pH 4.5 USP 34 acetate buffer, used to simulate the vaginal fluid, with a progressively decreasing volume in the 20-5 μl range (P100 micropipette, Eppendorf, Milan), are added to the sponges. Three tests are conducted on each sponge.

The quantity of liquid required for complete dissolution is under 1.0 ml.

Dissolution Time

The time required to dissolve the sponges is determined by chronometer after the addition to the sponges of the entire volume of pH 4.5 USP 34 acetate buffer required for dissolution. Three tests are performed for each sponge.

The dissolution times of the sponges are very fast, amounting to well under a minute.

The following experimental examples illustrate the invention.

### EXAMPLE 1

Formulation containing 200 μg of nitroglycerin, in the form of a sponge weighing 77.9 mg.

| | | Percentage weight |
|---|---|---|
| Ingredient | Weight (mg) | compared with total |
| Nitroglycerin | 0.2 | 0.26% |
| PVP 90 | 33.4 | 42.87% |
| PVP XL 10 | 11.7 | 15.02% |
| Mannitol | 32.6 | 41.85% |

The method for the preparation of a lyophilisate having the composition reported above is described below.

4 μl of a 5% w/w solution of nitroglycerin in propylene glycol, corresponding to 0.2 mg of nitroglycerin, is adsorbed on 11.7 mg of solid PVP XL 10, placed in an amber glass vial with a nominal volume of 5 ml, which is stoppered immediately after the addition of the medicament. 105 μl of bidistilled water is added to the mixture after 10 min to obtain a suspension of about 10% w/w in PVP XL 10. 556.7 mg of a 6% w/w aqueous solution of PVP 90 and 326 μl of a 10% w/w aqueous solution of mannitol are then added to said suspension.

The resulting mixture is freeze-dried.

### EXAMPLE 2

Formulation containing 200 μg of nitroglycerin.

Proceeding as reported in Example 1, a sponge-like freeze-dried formulation weighing 80.6 mg is obtained.

| | | Percentage weight |
|---|---|---|
| Ingredient | Weight (mg) | compared with total |
| Nitroglycerin | 0.2 | 0.25% |
| PVP 90 | 29.4 | 36.47% |
| PVP XL 10 | 10 | 12.41% |
| Mannitol | 41 | 50.87% |

1. A pharmaceutical composition comprising:
nitroglycerin from about 0.04 to about 0.3% w/w;
a mixture of crosslinked polyvinylpyrrolidone and non-crosslinked polyvinylpyrrolidone from about 30 to about 70% w/w; and
a cryoprotectant or stabilising agent from about 20 to about 60% w/w.

2. A composition according to claim 1 in form of a lyophilisate.

3. A composition according to claim 1, wherein the crosslinked polyvinylpyrrolidone is crospovidone.

4. A composition according to claim 3 wherein the crospovidone is PVP XL 10.

5. A composition according to claim 1, wherein the non-crosslinked polyvinylpyrrolidone is a polyvinylpyrrolidone having a molecular weight ranging from about 1,100,000 to about 1,500,000 daltons.

6. A composition according to claim 5 wherein the non-crosslinked polyvinylpyrrolidone has a molecular weight of about 1,300,000 daltons.

7. A composition according to claim 5 wherein the non-crosslinked polyvinylpyrrolidone is PVP 90.

8. A composition according to claim 1 wherein the weight ratio in the mixture between polyvinylpyrrolidone and non-crosslinked polyvinylpyrrolidone ranges from about 1:2 to about 1:4.

9. A composition according to claim 8 wherein the weight ratio between PVP XL 10 and PVP 90 ranges from about 1:2.2 to about 1:3.7.

10. A pharmaceutical composition according to claim 1, comprising:
nitroglycerin from about 0.05 to 0.28, preferably from about 0.24 to about 0.28% w/w;
PVP 90 from about 35.0 to about 45.0% w/w;
PVP XL 10 from about 10.0 to about 18.0% w/w; and
mannitol from about 40.0 to about 52.0% w/w.

11. A composition according to claim 1, wherein said composition is a freeze-dried single dosage form, containing from about 50 to about 200 μg of nitroglycerin.

12. A composition according to claim 11, wherein said composition is in a sponge-like form, and has a nitroglycerin concentration ranging from about 0.05 to about 0.3%.

13. A process for preparing a pharmaceutical composition, as defined in claim 1, comprising:
a) adsorbing nitroglycerin on a grade of solid cross-linked polyvinylpyrrolidone;
b) adding bidistilled water to the mixture obtained in step a);
c) adding volumes of aqueous solutions containing non-crosslinked polyvinylpyrrolidone and a cryoprotectant, respectively, to the aqueous suspension obtained in step b); and
d) freeze-drying the mixture thus obtained.

14. A process according to claim 13, wherein nitroglycerin is diluted in a C2-c5 glycol, preferably at about 5% w/w in propylene glycol.

15. A pharmaceutical composition as obtainable by the process of claim 13.
